(19) United States
(12) Patent Application Publication
(10) Pub. No.: US 2008/0166807 A1
(43) Pub. Date: Jul. 10, 2008

(54) Autologous progenitor stem cells of gastrointestinal origin

(76) Inventor: Padmanabhan P. Nair, Ellicott City, MD (US)

Correspondence Address: MISHRILAL JAIN, 11700 Old Yeorgetown Road, Unit 1501, North Bethesda, MD, 20852, US

(21) Appl. No.: 11/725,197

(22) Filed: Oct. 15, 2007

Related U.S. Application Data
(62) Division of application No. 11/059,112, filed on Feb. 16, 2005, now abandoned.

(51) Int. Cl. C12N 5/02

(52) U.S. Cl. 435/377; 435/378

(57) ABSTRACT

This invention relates to the discovery of autologous stem cells of gastrointestinal origin in fecal matter. More particularly, the invention relates to the isolation and propagation of said stem cells in continuous culture. Furthermore, the invention describes a method of directing and converting these gastrointestinal progenitor stem cells into immunoglobulin producing cells that secrete autologous antibodies to an antigen. In addition, the invention describes a method of isolating antibodies secreted by said immunoglobulin producing cells. The invention also describes a method of generating a lineage of antibody producing cells by growing said progenitor stem cells isolated from fecal matter on a feeder layer of tumor cells.

### FIELD OF INVENTION

This invention relates to the discovery of autologous stem cells of gastrointestinal origin in fecal matter. More particularly, the invention relates to the isolation and propagation of said stem cells in continuous culture in an unlimited, replicative state in vitro. Furthermore, the invention describes a method of directing and converting these gastrointestinal progenitor stem cells (GIP-C) into immunoglobulin producing cells that secrete autologous antibodies to an antigen.

### BACKGROUND OF INVENTION

The gastrointestinal epithelium undergoes constant and rapid renewal requiring a steady source of proliferating cells to maintain the turnover rate. The deep cryptal layers of the epithelium carry proliferating stem cells that form the source of these cells. These stem cells retain their functional competence over the lifetime of the individual. Tissue based histological studies show that these cells have no loss of cell density and rarely acquire age dependent genetic defects¹. Until now there has been no evidence to indicate the existence of this progenitor cell population in the exfoliated milieu of cells shed into the fecal stream. Although colonic cells can be recovered in a viable state from stool samples and examined for markers of gastrointestinal (GI) pathology² (U.S. Pat. No. 6,335,193), it has not heretofore been known that exfoliated progenitor stem cells of gastrointestinal origin are excreted in fecal matter and that these stem cells can be isolated and maintained in continuous culture in vitro.

### SUMMARY OF INVENTION

It is, therefore, an object of the present invention to isolate and characterize exfoliated progenitor stem cells from fecal matter.

Another object of the present invention is to provide a method for isolating progenitor stem cells from fecal matter, comprising the steps of:
(i) collecting a sample of fecal matter in SCSR-T medium;
(ii) dispersing the fecal sample in the SCSR-T medium;
(iii) sedimenting the cells present in the dispersed sample in step (ii) by layering the suspension over a medium of heavier density;
(iv) centrifuging the suspension in step (iii) to form a cellular band at the boundary with said heavier medium and combining cells present within said heavier medium and pellet; and
(v) culturing the cells obtained from step (iv) to selectively enlarge the population of stem cells present therein.

It is a further object of the present invention to describe a method for maintaining these stem cells in continuous culture in vitro.

It is yet another object of the present invention to show that these stem cells express IgA, IgA receptor, secretory component, IgG, IgG receptor, CD20, SSEA-1, SSEA-3, SSEA4, β-actin, cytokeratin 19 and bind jacalin.

It is an additional object of the present invention to describe a method for directing and converting these stem cells into immunoglobulin producing cells that secrete autologous antibodies to an antigen.

It is yet another object of the present invention to produce antibody secreting stem cells generated by allowing the said stem cells to grow on a feeder layer of tumor cells.

A further object of the present invention is to generate antibody from a lineage of stems cells derived from progenitor stem cells isolated from fecal matter. An additional object of the invention is to provide a composition for continuous culture of isolated progenitor stem cells obtained from fecal matter, comprising:
(i) 10%—fetal bovine serum in McCoy's 5A modified medium supplemented with 2 mM L-glutamine;
(ii)2.0-5.0 ml mesenchymal stem cell stimulatory supplement; and
(iii) 2.0-5.0 ml mesencult stem cell medium. Various other objects and advantages will become evident from the following detailed description of the invention.

### BRIEF DESCRIPTION OF DRAWINGS

The above and various other objects and advantages of the present invention will be better understood by reference to the accompanying drawings which are only illustrative and not limiting of the embodiments of the invention.

FIG. 1 shows phase contrast photomicrograph of GIP-C cells.

### DETAILED DESCRIPTION OF INVENTION

The present Invention is related to the characterization of exfoliated progenitor cells isolated from fecal matter, and to methods relating to their maintenance in continuous culture.

It should be noted that these exfoliated progenitor cells isolated from fecal matter are designated as “stem cells”, which term herein means that they are self-maintaining, have extensive self-renewing proliferative capacity and can be directed or converted to a differentiated derivative. Characterization of the isolated stem cells of the present invention is accomplished by well-established standard methodology including detection of certain cell surface markers through flow cytometry, nuclear staining techniques using propidium iodide, by the expression of epithelial lineage marker cytokeratin-19, or by the housekeeping gene β-actin through RT-PCR, and the like³⁻⁶.

The growth and maintenance of these stem cells in continuous culture is accomplished in a unique defined medium, both in the presence and absence of antibiotics, and by coating of plates with various epithelial attachment factors.

A subset of cells that has been differentially converted into a lineage of immunoglobulin secreting cells has also been accomplished.

It was discovered that these stem cells express at least the following cell surface markers: IgA, IgG, IgAR, IgGR, secretory component, CD8, CD20, SSEA1 (Stage Specific Embryonic Antigen 1), SSEA3, SSEA4, IgA+IgG, IgA+SC, IgA+SSEA1, and IgA+SSEA3⁷⁻¹¹. By combining nuclear staining with cell surface marker methodology, it was found that there are at least two different lineage-committed populations of cells. It was further established by isolating mRNA and expressing cytokeratin-19 and β-mactin through RT-PCR that these cells are intact and that there is a subpopulation committed to epithelial lineage.

The progenitor cells of the present invention grown in culture are mostly in suspension and when these cells were tested with five epithelial cell attachment factors, viz., collagen type IV, fibronectin, superfibronectin, ECM gel and laminin, it was discovered that a certain population of the these cells does attach itself to all the attachment matrices.

It should be understood that unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the methods and materials described herein are preferred. Unless mentioned otherwise, the techniques employed or contemplated herein are standard methodologies well known to one of ordinary skill in the art. The materials, methods and examples are only exemplary and not limiting.

### Methods and Materials

Isolation of Cells Containing GIP-C from Stool Samples.

Optimal mass of a fecal sample (generally about 0.5-1.0 gram) was collected at normal ambient temperature in a preweighed collection tube containing 15 ml of SCSR-T media (Noninvasive Technologies, 8170 Lark Brown Road, Suite 101, Elkridge. Md. 21075)) and glass beads. The sample was vortexed thoroughly and poured into a 7″×12″ doublesided filter bag (Tekmar-Dohrmann, Cat no. 10-0838-00). The sample was filtered through 40 μm nylon filter (BD-352340). The resuspended filtered sample from about 0.5 g of original fecal matter was removed and the volume was adjusted to 25 ml with SCSR-T media (NonInvasive Technologies). Ten ml of cushion solution, SCSR-C (NonInvasive Technologies) was used to underlay the sample without disturbing the top layer. The samples were spun for 10 minutes at 200 g. After discarding the supernatant the interface was carefully removed into a prelabelled 50 ml tube. The pellet and cushion above the pellet were placed in separate 50 ml tubes. The volume of the Pellet and Interface was adjusted to 15 ml by adding 1×PBS pH 7.2 and the samples were resuspended by inverting the tubes several times. The supernatant was carefully decanted after spinning the samples at about 2000 rpm, for about 10 minutes at about 4° C. (IEC Centra-8R Centrifuge). Another 15 ml of 1×PBS was added to the tubes and the cells were spun again at the conditions mentioned previously. The pellet was resuspended in 1 ml of 1×PBS (a cell count was obtained using a Z2 Beckman Coulter) and 250 μl aliquots of the sample was transferred into four 1.5 ml tubes. The cells were spun at about 1500 rpm, for about 10 minutes at about 4° C. The supernatant was aspirated and the pellets were resuspended in 50 μl of serum free cell freezing medium (Sigma C2639), and stored at −70° C. for archival purposes if they were not used for cell culture.

### Culture Conditions and Methodology

Cells were isolated from about 0.5 g of stool as described in the procedure above, and the final pellets were resuspended in 1 ml of 1×PBS pH 7.2 and counted using a Z2 Beckman Coulter counter. Two ml of Methocult SF (04436) and Mesencult (05401) stem cell media, purchased from Stemcell Technologies Inc, was added to four wells of a six well plate. An aliquot containing the number of cells desired from pellet and interface was added to each plate. The cells were maintained in a CO₂ incubator (5% CO₂) at about 37° C. for a week before the cells were subcultured, while observations were made everyday. To subculture the cells, 2 ml of sterile 1×PBS pH 7.4 was added to one of the two wells for pellet and interface. The wells were scraped and the contents were pippetted into 15 ml conical tubes. Another 5 ml of 1×PBS pH about 7.4 was added to the well to rinse, and the contents were added to 15 ml tube. The samples were spun at about 1500 rpm, for about 10 minutes at about 4° C. (IEC Centra—8R Centrifuge). After the supernatant was aspirated, the pellets were resuspended in 2 ml 1×PBS pH about 7.4. Then about 0.5 ml of the resuspended cells were added to each well containing the appropriate stem cell medium.

### Methocult SF Composition:

1%—Methylcellulose in Iscove's MDM
1%—Bovine Serum Albumin
10 μg/ml—Bovine Pancreatic Insulin
200 μg/ml Human transferrin (Iron-saturated)
3 U/ml—rh Erythropoietin
10 μl 2-Mercaptoethanol
2 mM—L-glutamine
50 ng/ml—rh Stem cell factor
20 ng/ml—rh GM-CSF
20 ng/ml—rh IL-3
20 ng/ml—rh IL-6
20 ng/ml—rh G-CSF

### Mesencult Media Composition:

Fetal bovine serum (10%) in McCoy's 5A Medium (modified) supplemented with L-glutamine (2 mM). Mesenchymal stem cell stimulatory supplements (human) (Stemcell Technologies Cat No. 05402) was added to the Mesencult media.

### Flow Cytometry

Flow cytometric studies were conducted for two main categories, namely surface antigen staining and nuclear staining. The surface antigen staining studies were further divided into three categories: direct staining, indirect staining and two color staining.

### Surface Antigen Staining

### Direct Staining Studies:

The following markers were tested using direct staining studies with antibodies at the appropriate dilution on the subcultured GIP-C cells derived from two separate sources grown in methocult and mesencult stemcell media. (a) The presence of IgA was tested using two different methods: (i) goat anti human IgA FITC (α chain specific) (Sigma, F2879) and (ii) Jacalin FITC from Artocarpus integrifolia (EY Laboratories, F6301-2). (b) Secretory component: FITC conjugated IgG fraction of goat polyclonal antiserum to human secretory component, free and bound (Nordic Immunology, 3913). (c) IgG: anti human IgG (y chain specific) PE conjugate developed in goat (Sigma, P8047). (d) FCR/IgG Receptor (IgGR): human IgG FITC (Sigma, F9636). (e) CD8: mouse monoclonal anti CD8 clone UCHT4 FITC conjugate (Sigma, F0772). (f) CD4: mouse monoclonal anti CD4 clone Q4120 FITC conjugate (Sigma, F1773). (g) CD19: mouse monoclonal antibody, clone 4G7 FITC conjugate (Becton Dickinson, 347543). (h) CD34: monoclonal antibody from mouse (Anti-HPCA-2) clone 8G12 FITC conjugate (Becton Dickinson, 348053). (i) CD45: monoclonal antibody from mouse, clone 2D1 FITC conjugate (Becton Dickinson, 347463). (j) CD45RA: monoclonal anti-human CD45RA FITC conjugate, clone F8-11-13 (Sigma, F1527). (k) CD54: monoclonal antibody from mouse, clone LB-2 PE conjugate (Becton Dickinson, 347977). (I) CD117: monoclonal antibody from mouse, clone 104D2, PE conjugate (Becton Dickinson, 340529).

The subcultured GIP-C cells from the two samples, A and B, that were stored in freezing media, at −70° C., were washed in 1 ml of 1×PBS 1% BSA. The cells were spun down at 1500 RPM for five minutes (IEC Centra—8R Centrifuge), aspirated and resuspended in 0.5 or 1 ml of PBS 1% BSA depending on the cell counts. Flow Cytometric studies were conducted with 100,000-200,000 cells which were aliquoted into 5 ml polystyrene round bottom tubes (BD Falcon, 352054) and the volume was adjusted to 100 μl with 1×PBS 1% BSA. Between 10 μl-20 μl of antibody, diluted in PBS containing 1% BSA was added to each sample. The samples were then incubated for an hour at 37° C. while gently mixing. They were then washed twice by adding 2 ml of cold 1×PBS 1% BSA per tube and mixed against the side of the rack, and spun at 2000 RPM for five minutes at 4° C. (IEC Centra—8R Centrifuge), and the supernatant was removed. The cells were then resuspended in 1 ml of 6% paraformaldehyde. The samples were stored at 4° C. until analyzed.

### Indirect Staining Studies:

The following markers were used for indirect staining studies with subcultured GIP-C cells (A and B) using antibodies with appropriate dilutions. The antibodies for the markers tested were added in the following order; (a) IgA Receptor (IgAR): monoclonal anti human IgA (α chain specific), clone GA-112 (Sigma, I0636)+Human IgA pure from human colostrum (Sigma, I1010)+goat anti human IgA FITC. (b) CD20: monoclonal anti human CD20 purified mouse immunoglobulin (Sigma, C8080) +anti mouse IgG (whole molecule) FITC conjugate developed in rabbit (Sigma, F9137). (c) SSEA1: mouse anti SSEA1 monoclonal antibody clone MC480, immunogen: F9 teratocarcinoma stemcells (X-irradiated), (MAB4301 Chemicon)+anti mouse IgG FITC conjugate. (d) SSEA3: mouse anti SSEA3 monoclonal antibody, clone MC 631, immunogen: 4-8 cell stage mouse embryos, (MAB4303 Chemicon)+anti mouse IgG FITC conjugate. (e) SSEA4: mouse anti SSEA4 monoclonal antibody clone MC-813-70, immunogen: human embryonal carcinoma cell line 210Ep, (MAB4304 Chemicon)+anti mouse IgG FITC conjugate.

The procedure of indirect staining was similar to that of direct staining, the only difference being the length of the incubation period, which was 30 minutes at 37° C. for each antibody that was added to the cells.

### Two Color Staining Studies:

The following markers were used for two color staining studies with subcultured GIP-C cells initially from both A and B methocult and mesencult samples, but later only from B mesencult pellet and interface cells from passages 38 and 40. The antibodies for the markers tested were added in the following order: (Note: the first two markers were initially tested with A and B samples, added during separate incubations at 37° C. for 30 minutes) (a) IgA (FITC)+SC (PE): goat anti human IgA FITC+mouse monoclonal anti-human SC (Sigma, 16635)+Rabbit anti mouse IgG (whole molecule) PE (Sigma, P0313). (b) IgA (jacalin FITC)+SC (PE): jacalin FITC (Artocarpus integrifolia)+mouse monoclonal anti-human SC+Rabbit anti mouse IgG (whole molecule) PE. (c) IgA FITC+SSEA1 PE: goat anti human IgA FITC (a chain specific)+mouse anti SSEA1 monoclonal antibody clone MC480+Rabbit anti mouse IgG (whole molecule) PE. (d) IgG (PE)+SSEA1 (FITC): anti human IgG (y chain specific) PE conjugate+mouse anti SSEA1 monoclonal antibody clone MC480+anti mouse IgG (whole molecule) FITC. (e) IgA (FITC)+IgG (PE): goat anti human IgA FITC+anti human IgG PE. (f) IgG (PE)+IgA (FITC): anti human IgG PE+goat anti human IgA FITC. (g) IgA (FITC)+SSEA3 (PE): goat anti human IgA FITC+rat anti SSEA3 monoclonal antibody, clone MC 631+rabbit anti mouse IgG (whole molecule) PE. (h) IgG (PE)+SSEA3 (FITC): anti human IgG PE+rat anti SSEA3 monoclonal antibody, clone MC 631+anti mouse IgG FITC. (i) IgA (FITC)+SSEA4 (PE): goat anti human IgA FITC+mouse anti SSEA4 monoclonal antibody clone MC-813-70+rabbit anti mouse IgG (whole molecule) PE. (j) IgG (PE)+SSEA4 (FITC): anti human IgG PE+mouse anti SSEA4 monoclonal antibody clone MC-813-70+anti mouse IgG (whole molecule) FITC. (k) IgA (FITC) +CD20 (PE): goat anti human IgA+monoclonal anti human CD20+rabbit anti mouse IgG (whole molecule) PE. The antibodies were added in the given order and incubated separately at 37° C. for 30 minutes.

Some of the markers tested above were added together and incubated overnight at 4° C. as well. They were as follows: (a) IgA (FITC)+IgG (PE); (b) IgA (FITC)+CD20 (PE); (c) IgA (FITC)+SSEA3 (PE). The markers that needed the addition of a secondary antibody carrying a FITC or PE label were added after the first wash, after the overnight incubation, and then incubated for a half hour at 37° C.

### Nuclear Staining Studies

The nuclear staining studies were done Initially by adding propidium iodide staining solution to the subcultured GIP-C cells and later to subcultured GIP-C cells that were initially stained with fluorescently-labelled antibodies. The procedures for both the studies were as follows:

Preliminary Studies using Propidium Iodide with Subcultured GIP-C Cells

Propidium iodide (BD Biosciences) was dissolved in PBS (pH 7.4) at the concentration of 50 μg/ml. Two tubes of one million GIP-C cells from five different subcultured samples were used. The samples were as follows.

Five different subcultured samples were chosen for this study: C: methocult pellet, A: methocult interface, B: methocult pellet, E: mesencult pellet and D: methocult interface. The cells were scraped and washed with PBS pH 7.4 and a cell count was obtained. The cells were spun down and then resuspended in PBS+1% BSA. Two tubes of one million GIP-C cells were obtained from each sample. Propidium iodide (50 μl of 50 μg/ml) was added to one tube of each sample to a 0.7 ml cell suspension the total volume being 950 μl. NP40 (200 μl, 0.5% in PBS 7.2) was added to the other set of tubes of GIP-C cells and they were put on Ice for 15 minutes. Propidium iodide (50 μl of 50 μg/ml) was added to the cells in NP40. Both sets of cell were run on the flow cytometer after vortexing the samples.

Propidium Iodide with Stained Subcultured GIP-C Cells

Another set of propidium iodide studies were done with stained GIP-C cells. Propidium iodide (10 μl of 50 μg/ml) was added to GIP-C cells stained with the following markers: (a) IgG (PE)+IgA (FITC), (b) IgA (FITC), (c) IgA (FITC)+SSEA1 (PE), (d) IgG (PE)+SSEA1 (FITC), (f) IgA (FITC)+SSEA3 (PE), (g) IgG PE+SSEA3 (FITC), (h) IgA (FITC)+SSEA4 (PE), (i) IgG (PE)+SSEA4 (FITC), (i) IgA (FITC)+CD20 (PE), (j) IgA (FITC)+IgG (PE). The antibodies for the above mentioned markers were added and incubated individually at 37° C. for a half hour period.

A third set of propidium iodide studies were done where the primary antibodies for two color markers were added together and incubated at 4° C. overnight initially and after a wash the secondary antibody was added and incubated at 37° C. for a half hour before being fixed. Propidium iodide (10 μl of 50 μg/ml) was added to the following markers: (a) IgA (FITC)+IgG (PE), (b) IgA (FITC)+CD20 (PE) and (c) IgA (FITC)+SSEA3 (PE).

Isolation of mRNA from Subcultured GIP-C Cells.

mRNA from subcultured GIP-C cells were isolated using the procedure described by Nair etal². Also a PCR was run testing the presence of β-actin (housekeeping gene), and cytokeratin 19 (marker for epithelial lineage) primers. The sequences of the primers used were as follows:

| | |
|---|---|
| β-actin | 5′ TCACCAACTGGGACGACATG |
| β-actin | 3′ ATGTCACGCACGATTTCCCG |
| Keratin19 | 5′ ATCCTGAGTGACATGCGAAGC |
| Keratin19 | 3′ CATGAGCCGCTGGTACTCCTG |

Antibiotic Studies with Subcultured GIP-C Cells

A 100× antibiotic/antimycotic mixture was obtained from Sigma (A5955). Six different concentrations of antibiotic/antimycotic were used in mesencult media. The concentrations used were as follows: 0×, ⅕×, ⅖×, ⅗×, ⅘× and 1×. Half a million cells of specimen A mesencult pellet and interface cells were added to 1.5 ml of different concentrations of antibiotic/mycotic mixture in mesencult media. The cells were incubated at 37° C. for a week, and were observed daily. Initially triplets were made of the same concentrations, but a week later the triplets of each concentrations were combined and subcultured and resuspended in mesencult media containing the appropriate concentrations of antibiotic/mycotic mixture. Cell counts were obtained from all of the subcultured samples.

In order to observe the cells under the hemocytometer, the subcultured GIP-C cells which were exposed to the antibiotic/mycotic mixture had to be washed initially. The procedure is as follows: 0.5 ml of GIP-C cells were obtained from 0× and 1× samples. A solution containing 50 mg of NCL006 (NonInvasive Technologies) in 5 ml of mesencult media was made. One ml of the mixture was added to 0.5 ml of the GIP-C cells and mixed. After waiting for 5 minutes the cells were spun for 5 minutes at 2000 RPM at 4° C. (IEC Centra—8R Centrifuge). This step was repeated once again. The pellet was resuspended in 0.5 ml of mesencult media, and added to another 0.5 ml of media with the appropriate concentration of antibiotic/mycotic mixture. A small aliquot of cells were taken and observed under the hemocytometer, while the rest of the GIP-C cells were incubated at 37° C. in the antibiotic/mycotic media.

Study of Attachment Factors with Subcultured GIP-C Cells
Coating the Wells with Attachment Factors:

Two of the six well plates were coated with the following attachment factors: Collagen type IV, Fibronectin, Superfibronectin, ECM gel and Laminin. Collagen type IV (2 mg/ml) was made by dissolving 0.75 mg of collagen in 375 μl of sterilized 0.25% acetic acid. Collagen type IV (187.5 μl) was used to coat two wells and Incubated overnight at 2-8° C. Fibronectin (250 μl of 10 μg/ml) was used to coat each well and air-dried at room temperature for 45 minutes. Superfibronectin (250 μl of 5 μg/ml) was used to coat each well and incubated at 37° C. for two hours. The wells were then washed twice with 100 μl of PBS, and left to air dry in a laminar flow hood. ECM gel was thawed overnight before use; 250 μl of ECM gel was mixed with 250 μl of cold DMEM medium before using 250 μl of the mixture to coat precooled wells. The ECM gel was then left for five minutes at 20° C. for the gel to polymerize. An aliquot of 200 μg/200 μl of laminin was thawed at 4° C. for 2-4 hrs before making a dilution using 36 μl of stock solution and diluting it with 464 μl of sterile PBS. Laminin (250 μl, 18 μg/well) was used to coat each well. The plates with laminin were then air-dried for 45 minutes. All of the plates containing the attachment factors were sterilized overnight by exposure to UV light in a sterile tissue culture hood.

### Preparing the RPMI 1640 Media for Attachment Factor Study:

RPMI 1640 media (250 ml) was obtained from Invitrogen (11875-085), to which the following supplements were added: 100 ng/ml of cholera-toxin (Sigma, C8052), 500 ng/ml of hydrocortisone (Sigma, H0888), 5% of heat inactivated horse serum (Gibco, 16050-130) and 25 ml of Mesenchymal supplement (Stemcell technologies, 05402). After the media was filter sterilized and aliquots of 26 ml were made, they were stored at 4° C.

RPMI 1640 media with all the supplements was warmed at 37° C. Two ml of the media was added to each well containing the attachment factors along with 3 control wells which contained no attachment factors. Cells previously subcultured in mesencult medium were pelleted and divided into 100,000 cells per well and incubated at 37° C. The cells were observed under a phase-contrast microscope. Once, every two days the top layer containing the unattached GIP-C cells was removed, washed and resuspended in freezing media. Two ml of RPMI 1640 media with the supplements was added to the wells and incubated at 37° C.

### Results

A number of markers were tested on the subcultured progenitor cells using flow cytometry. The following table displays the statistics on the percentage of progenitor cells expressing the markers tested:

TABLE 1

| Markers | Passage No. | N | Mean | SEM |
|---|---|---|---|---|
| IgA FITC | 12 & 16 | 16 | 48.9 | 3.3 |
| SC FITC | 12, 13, 15, & 16 | 32 | 27.9 | 2.6 |
| IgG PE | 13 & 15 | 16 | 45.5 | 2.3 |
| IgAR FITC | 13 | 8 | 58.5 | 5.0 |
| FcR/IgGR FITC | 13 | 8 | 65.9 | 5.4 |
| CD 8 FITC | 18 | 8 | 1.3 | 0.2 |
| CD 20 FITC | 22, 23, 40 | 18 | 38.5 | 3.0 |
| SSEA1 FITC | 22, 23, 38 & 40 | 20 | 33.7 | 2.5 |
| SSEA3 FITC | 22, 23, 38 & 40 | 20 | 40.4 | 2.7 |
| SSEA4 FITC | 22, 23 & 38 | 18 | 38.9 | 2.7 |
| * SC (PE) + IgA (FITC) | 12 & 16 | 16 | | |
| LR quadrant IgA FITC | | | 30.1 | 2.2 |
| UL quadrant SC PE | | | 1.0 | 0.4 |
| UR quadrant IgA + SC | | | 6.7 | 2.0 |
| ** IgA (FITC) + SC (PE) | 13 & 16 | 16 | | |
| LR quadrant IgA FITC | | | 11.4 | 2.6 |
| UL quadrant SC PE | | | 3.4 | 0.8 |
| UR quadrant IgA + SC | | | 2.7 | 0.8 |
| IgA Jacalin (FITC) | 15 | 8 | 31.9 | 3.1 |
| * SC (PE) + IgA Jacalin (FITC) | 15 & 16 | 16 | | |
| LR quadrant IgA jac FITC | | | 14.3 | 2.1 |
| UL quadrant SC PE | | | 0.7 | 0.3 |
| UR quadrant IgA + SC | | | 18.6 | 4.3 |
| ** IgA Jacalin (FITC) + SC (PE) | 15 & 16 | 16 | | |
| LR quadrant IgA jac FITC | | | 6.6 | 2.1 |
| UL quadrant SC PE | | | 2.3 | 0.6 |
| UR quadrant IgA + SC | | | 17.4 | 4.2 |
| IgA (FITC) + IgG (PE) | 38 & 40 | 4 | | |
| LR quadrant IgA FITC | | | 22.5 | 1.7 |
| UL quadrant IgG PE | | | 6.2 | 0.5 |
| UR quadrant IgA + IgG | | | 13.0 | 3.0 |
| IgA (FITC) + CD20 (PE) | 40 | 4 | | |
| LR quadrant IgA FITC | | | 2.8 | 1.8 |
| UL quadrant CD20 PE | | | 1.5 | 0.3 |
| UR quadrant IgA + CD20 | | | −1.7 | 1.8 |
| IgA (FITC) + SSEA1 (PE) | 38 & 40 | 4 | | |
| LR quadrant IgA FITC | | | 8.2 | 1.6 |
| UL quadrant SSEA1 PE | | | 1.2 | 0.4 |
| UR quadrant IgA + SSEA1 | | | 5.8 | 2.1 |
| IgA (FITC) + SSEA3 (PE) | 38 | 2 | | |
| LR quadrant IgA FITC | | | 7.5 | 6.0 |
| UL quadrant SSEA3 PE | | | 1.1 | 0.7 |
| UR quadrant IgA + SSEA3 | | | 10.9 | 1.3 |
| IgA (FITC) + SSEA4 (PE) | 38 | 2 | | |
| LR quadrant IgA FITC | | | 0.4 | 1.1 |
| UL quadrant SSEA4 PE | | | 3.8 | 1.6 |
| UR quadrant IgA + SSEA4 | | | 15.9 | 0.3 |
| IgG (PE) + SSEA1 (FITC) | 38 | 2 | | |
| LR quadrant SSEA1 FITC | | | 23.1 | 3.0 |
| UL quadrant IgG PE | | | 0.1 | 0.0 |
| UR quadrant IgG + SSEA1 | | | 38.3 | 9.6 |
| IgG (PE) + SSEA3 (FITC) | 38 | 2 | | |
| LR quadrant SSEA3 FITC | | | 32.7 | 5.2 |
| UL quadrant IgG PE | | | 0.5 | 0.0 |
| UR quandrant IgG + SSEA3 | | | 18.2 | 2.6 |

* The order of the antibodies added was SC (PE) and IgA (FITC).
** The order of the antibodies added was IgA (FITC) and SC (PE).
SEM: Standard Error of Mean
N: Number of replicates

Flow cytometric studies with progenitor cells were negative for the expression of the following markers: CD19, CD34, CD45, CD45RA, CD54 and CD117. There was a small population of cells containing CD8 (1.3%), a T-cell marker, and about 38.5% of cells containing CD20, a B-cell marker. Coexpession studies of IgA+SC (secretory component) were performed using anti IgA antibody as well as jacalin FITC (which recognizes IgA). Without being bound to any theory, it is hypothesized that the antibody against IgA initiated a biological activity in cells which led to masking or endocytosis or even reorientation of IgA in the membrane which produced low detection levels of free IgA (this phenomenon was also detected with other combination studies discussed below). While for non-antibody marker (Jacalin FITC) such biological activity was not elicited, these studies along with backgating exercises and histogram analysis showed that most of the progenitor cells coexpressed IgA+SC on the same cell. The existence of cells containing only IgA as well as secretory component was also observed. Two-color flow cytometric studies to elicit coexpression of IgA+IgG showed the presence of four different populations of cells: cells with IgA, cells with IgG, cells with IgA+IgG, and cells containing neither IgA nor IgG. Similar two-color studies of IgA and IgG with different Stage Specific Embryonic Antigens indicated that there might be lineage-directed changes in which some stem cells are transformed into immunoglobulin secreting cells while others continue dividing thereby maintaining a pool of progenitor cells available for lineage directed differentiation under appropriate conditions. When IgA was combined with CD20, the CD20 appeared to hinder the IgA binding to the cells, indicating an interaction that results in release of IgA+CD20 into supernatant.

Propidium iodide studies demonstrated that all the cells were nucleated, but not all nucleated cells necessarily carried the cell surface markers which were tested. When flow cytometry studies were combined with nuclear staining studies containing propidium iodide an upward shift in the population from the unstained region was observed. This was parallel to the shift seen in the LR quadrant events. When propidium iodide was added to cells stained with the three Stage Specific Embryonic Antigens (1, 3 and 4), about 5% of cells were nucleated and carried the marker for SSEA1 and 4, whereas, for SSEA3, 20% of the cells were nucleated and carried the marker. This suggested that there might be two populations for Stage Specific Embryonic Antigens in existence, one of which (SSEA3) was older than SSEA1 and SSEA4. This could indicate the existence of more nucleated cells in the SSEA3 population. For the next set of combination studies with IgA or IgG with SSEAs, a similar shift was observed when propidium iodide was added. This appearance of two distinct populations of cells indicated that there existed cells with different amounts of DNA. This phenomenon was also seen with IgA+CD20 when propidium iodide was added.

Expression of β-actin and Cytokeratin 19 by the Subcultured GIP-C Cells

mRNA was isolated from subcultured progenitor cells and then RT-PCR was performed to see if the cells expressed β-actin and cytokeratin 19.

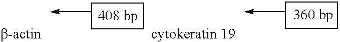

The subcultured cells expressed both β-actin and cytokeratin 19 proving that these cells were eukaryotic, some of which were of epithelial lineage.

Antibiotic/Antimycotic Studies with Subcultured GIP-C Cells.

It is a generally recognized that cells lining the large bowel (colon) are in constant contact with a large microbial population and are adapted to this environment. In a similar manner GIP-C cells also had high levels of bacteria associated with them, a condition that persisted even when the GIP-C cells were being grown in continuous culture. To observe the effect of antibiotics on the growth of these progenitor GIP-C cells, they were grown in culture with varying concentrations of antibiotic/antimycotic mixture. The cell counts taken at different times were as follows:

TABLE 2

| | | | 2nd subculture |
|---|---|---|---|
| | | 1st subculture | after washing w/ |
| Antibiotic | Initial Counts | 2-5 μm | NCL006 2-5 μm |
| concentrations | (Cells/ml) | (Cells/ml) | (Cells/ml) |
| 0x | 8.60E+06 | 1.54E+07 | 1.00E+07 |
| 1/5x | | 8.11E+06 | 2.98E+06 |
| 2/5x | | 5.13E+06 | 3.98E+06 |
| 3/5x | | 1.23E+06 | 2.26E+06 |
| 4/5x | | 3.57E+06 | 1.21E+06 |
| 1x | | 5.17E+06 | 2.18E+06 |

There was no consistent trend indicating a drop in the cell counts with increasing concentrations of antibiotic/antimycotic mixture. NCL006 (Noninvasive Technologies), 0.5% in PBS was used to wash the cells and clear the mucous before viewing the cells under a phase contrast microscope. Although the number of GIP-C cells had decreased considerably after each subculture, they still seemed to be growing and dividing in the presence of antibiotic/antimycotic mixture. There were some remnants of bacteria left in the higher antibiotic concentrations, but the media was mostly clear. At higher concentrations of antibiotic/antimycotic mixture GIP-C cells appeared to be larger in size. Cell counts were taken of GIP-C cells in sizes ranging from 1-3 μm, 2-5 μm, and 5-8 μm from the second subculture after washing with NCL006. The counts were as follows:

TABLE 3

| | Cell counts from 2nd subculture after | | |
|---|---|---|---|
| | washing w/NCL006 | | |
| Antibiotic | 1-3 μm | 2-5 μm | 5-8 μm |
| concentrations | (Cells/ml) | (Cells/ml) | (Cells/ml) |
| 0x | 6.23E+06 | 1.00E+07 | 972634 |
| 1/5x | 3.35E+08 | 2.98E+06 | 235170 |
| 2/5x | 796683 | 3.98E+06 | 212256 |
| 3/5x | 1.79E+08 | 2.26E+06 | 299088 |
| 4/5x | 2.60E+08 | 1.21E+06 | 185322 |
| 1x | 3.49E+06 | 2.18E+06 | 171252 |

The cell count seems to decrease as the size range increases from 1-3 μm to 2-5 μm to 5-8 μm.

### Attachment Factors Study

The gastrointestinal progenitor cells in culture were found mostly in suspension in the culture media. In order to test the adherence of these cells a study was conducted where they were plated on tissue culture wells coated with each of the five different epithelial cell attachment factors: collagen type IV, laminin, superfibronectin, fibronectin, and ECM gel. There were three control wells with no attachment factors in them. The cell counts obtained were as follows:

TABLE 4

| | Cell counts in | | Cell counts of Attached | |
|---|---|---|---|---|
| | Supernatant (Cells/ml) | | cells (Cells/ml) | |
| Attachment factors | 2-5 μm | 5-8 μm | 2-5 μm | 5-8 μm |
| Control 1 | 4.94E+07 | 162810 | | |
| Control 2 | 1.51E+07 | 141504 | | |
| Control 3 | 2.92E+07 | 143916 | | |
| Collagen type IV | 1.60E+07 | 149544 | 1.19E+06 | NA |
| (well 1) | | | | |
| Collagen type IV | 1.22E+07 | 65526 | 2.25E+06 | 57486 |
| (well 2) | | | | |
| Laminin (well 1) | 4.09E+07 | 90048 | 974448 | 40602 |
| Laminin (well 2) | 3.04E+07 | 84822 | 643602 | 43416 |
| Superfibronectin | 3.96E+07 | 67536 | 1.62E+06 | 14874 |
| (well 1) | | | | |
| Superfibronectin | 5.06E+07 | 54672 | 1.35E+06 | 52260 |
| (well 2) | | | | |
| Fibronectin (well 1) | 2.99E+07 | 68340 | NA | 52260 |
| Fibronectin (well 2) | 1.04E+06 | 63918 | 68340 | 20502 |
| ECM gel (well 1) | 2.94E+07 | 245220 | 1.32E+06 | 24924 |
| ECM gel (well 2) | 1.28E+07 | 54672 | 764604 | 53064 |

Even though most attachment factors seem to attach the cells, certain attachment factors such as collagen type IV, superfibronectin and ECM gel seemed to be better at attaching cells from 2-5 μm. As seen from the above data, there were also some cells of 5-8 μm that were attached to the five different attachment factors. It seemed that a certain population of cells was attached and others were in suspension, and the degree of attachment varied with the different attachment factors used.

### Culturing GIP-C Cells on Feeder Layer of Mitomycin C Treated HT-29 Cells for Inducing Immunoglobulin Producing Lineage

Preparation of Feeder Layer:
1. Grow HT-29 cells in McCoy 5A medium containing 10% FBS to confluency
2. Remove medium and wash cells with PBS −2 times
3. Add 15 ml of 10 ng/ml Mitomycin C stock solution to each flask and swirl to cover surface
4. Incubate at 37° C. for 3 hours
5. After incubation, aspirate off the Mitomycin C and wash 3 times with 20 ml of PBS
6. Add trypsin EDTA to remove the cells and add McCoy 5A medium containing 10% Fetal bovine serum to stop the trypsin action
7. Wash the cells three times with 20 ml of PBS at 2000 rpm for 10 min.
8. Check the cell count and suspend the cells in medium adjusting the density to about 3.5×10⁵ cells/ ml and put them in a tissue culture flask as follows: (Note: Cells should not be frozen after Mitomycin C treatment.)

### Culture of GIP-C Cells on Feeder Layer:

1. Seed the Mitomycin C treated cells (at about 3.5×10⁵ cells/ ml) in T-75 flask
2. Allow the cells to attach for at least 2 hrs or preferably overnight
3. Take frozen GIP-C cells at −70° C., which have already been grown in mesencult medium from interface and pellet fractions
4. Wash the GIP-C in cold PBS at 2000 rpm for 10 min
5. Seed these cultured GIP-C cells in 2 flasks/in RPMI-1640 medium containing 5% horse serum (HS) (heat-inactivated and preadsorbed IgG) from each fraction of interface and pellet. Also, take 2 flasks of mitomycin C treated HT-29 cells with complete RPM I 1640 medium alone as control.
6. Grow the cells for one week at 37° C. in 5% CO₂ without changing or adding the medium.

Mitomycin C Stock: (It is Toxic, Wear Gloves and Use Caution when Handling)
Prepare 1 mg/ml of PBS, store the solution in dark by covering the tube with aluminum foil and use within 2 weeks.
Diluted Stock #1 (10 μg/ml): Take 20 μl of stock (1 mg/ml), and add 2 ml of medium.
Diluted Stock #2 (10 ng/ml): Take 100 μl of diluted stock #1, and add 100 ml of medium.

TABLE 5
Modified RPMI-1640 medium used for transit
of GIP-C cells into IgG secreting lineage

| | | Final | | | | |
|---|---|---|---|---|---|---|
| | Stock solution | conc. | 10 ml | 50 ml. | 100 ml | 250 ml |
| 1 | RPMI-1640 medium | 83% | 7.8 ml | 39 ml | 78 ml | 195 ml |
| 2 | Glutamax (200 mM) | 2 mM | 100 μl | 500 μl | 1 ml | 2.5 ml |
| 3 | Horse serum or Human | 5% | 1 ml | 5 ml | 10 ml | 25 ml |
| | AB serum (50%, Heat | | | | | |
| | inactivated and | | | | | |
| | preadsorbed on Protein A | | | | | |
| | agarose) | | | | | |
| 4 | Mesenchymal supplement | 10% | 1 ml | 5 ml | 10 ml | 25 ml |
| 5 | Hydrocortisone(50 μg/ml) | 500 | 100 μl | 500 μl | 1 ml | 2.5 ml. |
| | | ng/ml | | | | |

### Stock Solutions:

1. Glutamax (200 mM): Aliquot 2.5 ml/vial and freeze at −20° C.
2. Horse serum:
   i) Heat Inactivation: Heat inactivate the HS by incubating at 56° C. for 30 min. Make aliquots of 12.5 ml in 15 ml sterile tubes.
   ii) Removal of IgG:
      a) Dilute HS to 50% by mixing 1 volume of HS with 1 volume of RPMI-1640 medium.
      b) Adsorb 3 volumes of 50% HS with 1 volume of protein A agarose packed beads.
      c) Stir at 4° C. overnight.
      d) Centrifuge at 4500 rpm for 10 min. Remove the supernatant and use it as preadsorbed HS.
      e) Regenerate protein A agarose according to the protocol.
1. Hydrocortisone (50 μg/ml):
   i) Add 5 ml of absolute ethanol to 5 mg of product, gently swirl to dissolve.
   ii) Prepare 25 aliquots of 200 μl each and store at −20° C.
   iii) Just before use, take 100 μl of stock solution and add 1.9 ml of RPMI 1640 medium.

TABLE 6
Amount of IgG eluted from GIP-C grown on feeder
layer of Mitomycin C treated HT-29 cells

| Type of | IgG eluted (mg/ml of culture supernatant) |
|---|---|
| Medium | Mitomycin C treated HT-29 cells + GIP-C |
| Medium 1 | 0.37 |
| Medium 2 | 0.33 |
| Medium 3 | 0.28 |
| Medium 4 | 0.01 |
| Medium 5 | 0.57 |

Medium 1: Modified RPMI 1640 medium without hydrocortisone
Medium 2: Modified RPMI 1640 medium without Mesencult supplement
Medium 3: Modified RPMI 1640 medium without hydrocortisone and Mesencult
Medium 4. Complete Modified RPMI medium with human AB serum
Medium 5: Complete Modified RPMI 1640 medium with horse serum

Isolation of IgG from Culture Medium of GIP-C
2. Hydrate 1 g of Protein A agarose (cat.# P-0932) In about 20 ml of phosphate buffer A. It should swell to about 4 ml packed volume, wash 2 times with 20 ml of phosphate buffer.
3. Resuspend 4 ml of beads in 4 ml of Buffer A (double the packed volume)
4. To each of 4 tubes containing filtered culture supernatant, add 1 ml of bead suspension from above (500 μl of packed volume or about 8 equal parts) to each tube, mix the beads well with the culture supernatant. Leave 8 tubes on the rocker platform for 2 hrs at room temp. Make sure that beads mix well with the culture supernatant well.
5. Centrifuge samples at 5000 rpm for 5 min at room temp.
6. Remove supernatant, combine supernatants from all 3 tubes of test interface, 3 tubes of test pellet and 2 tubes of control separately, save it as Wash #1 Interface, and Wash #1 pellet. Wash the beads with 10 ml of Buffer A by repeating step # 4 twice,
   Save the wash both times, separately as Wash #2 and Wash #3
7. Read absorbance at 280 nm for all 3 washes, absorbance in Wash #3 must be zero
8. Suspend the beads in about 1 ml of buffer A each, and pool them in one tube.
9. Remove supernatant and suspend the beads in 750 μl buffer B, pH 4.0 per tube, leave the tubes at RT for 15 min, mixing tubes 2-3 times during elution
10. Centrifuge at 5000 rpm for 5 min at RT
11. Remove the supernatant from each and collect it in a clean tube (Eluate 1)
12. Neutralize the eluate using prestandardised volume of 1 M Tris HCl, pH 9.0 (˜28 μl of 1 M TrisHCl/750 μl)
13. Repeat steps #7-#9 once again using 750 μl of Buffer B, pH 4.0. Neutralize using 1M Tris HCl, pH 9.0 as above (Eluate 2)
14. Check the pH at this point to make sure that pH is close to 7.0
15. Read absorbance at 280 nm for Eluate 1 and 2 from interface, and pellet (use BSA as standard @1 mg/ml).
16. Dialyze against sterile 0.9% NaCl and concentrate to the required volume 17. Centrifuge at 5000 rpm for 10 min.

Example embodiments of the present invention have now been described in accordance with the above advantages. It will be appreciated that these examples are merely illustrative of the invention and not limitations thereof. Many variations and modifications will be apparent to those skilled in the art and all such modifications and variations are included within the purview and scope of the appended claims.

### REFERENCES

1. Marshman E, Booth C, Potten C. The intestinal epithelial stem cell. BioEssays 2002, 24: 91-98.
2. Nair P, Lagerholm S, Dutta S, Shami S, Davis K, Ma S, Malayeri M. Coprocytobiology. J Clin Gastroenterology 2003, 36 (Suppl. 1): S84-S93.
3. Vacanti M P, Roy A, Cortiella J, Bonassar L, Vacanti C A Identification and initial characterization of spore-like cells in adult mammals J. Cell Biochem, 2001, 80: 455-460.
4. Shimizu M, Minakuchi K, Tsuda A, Hiroi T, Tanaka N, Koga J, Kiyono H. Role of stem cell factor and c-Kit signaling in regulation of fetal intestinal epithelial cell adhesion to fibronectin. Exp Cell Res 2001, 266: 311-322.
5. Kruger G M, Mosher J T, Bixby S, Joseph N, Iwashita T, Morrison S J. Neural crest stem cells persist in the adult gut but undergo changes in self-renewal, neuronal subtype potential, and factor responsiveness. Neuron, 2002, 35: 657-669.
6. Schon E A, Tales from the crypt. J Clin Invest 2003, 112: 1351-1360
7. Shambloft M J, Axelman J, Wang S, Bugg E M, Littlefield J W, Donovan P J, Blumenthal P D, Huggins G R, Gearhart J D. Derivation of pluripotent stem cells from cultured human primordial germ cells. Proc Natl Acad Sci USA 1998, 95:13726-13731.
8. Solter D, Knowles B B. Monoclonal antibody defining a stage-specific embryonic antigen (SSEA-1). Proc Natl Acad Sci USA 1978, 75: 5565-5569.
9. Thomson J A, Itskovitz-Eldor J, Shapiro S S, Waknitz M A, Swiergiel J J, Marshall V S, Jones J M. Embryonic stem cell lines derived from human blastocysts. Science 1998, 282:1145-1147.
10. Draper J S, Pigott C, Thomson J A, Andrews P W. Surface antigens of human embryonic stem cells: changes upon differentiation in culture. J Anat 2002, 200: 249-258.
11. Vassilieva S, Guan K, Pich U, Wobus A M. Establishment of SSEA-1 and Oct-4-expressing rat embryonic stem-like cell lines and effects of cytokines of the IL-6 family on clonal growth. Exp Cell Res 2000, 258:361-373.

1-15. (canceled)

16. A method for isolating progenitor stem cells from fecal matter, comprising the steps of:
(i) collecting a sample of fecal matter in somatic cell sampling and recovery transport (SCSR-T) medium;
(ii) dispersing the fecal sample in the SCSR-T medium;
(iii) sedimenting the cells present in the dispersed sample in step (ii) by layering the suspension over a medium of density heavier than that of the suspension;
(iv) centrifuging the suspension in step (iii) to form a pellet and a cellular band at the boundary with said heavier medium, and combining cells present within said heavier medium and the pellet; then
(v) exposing the cells obtained in step (iv) to a medium that allows only progenitor stem cells present therein to propagate in continuous culture in an unlimited, replicative state; and then
(vi) maintaining progenitor stem cells obtained from step (v) in continuous replicative state.

17-19. (canceled)

20. The method of claim 16, wherein said medium for continuous culture comprises per volume of 6-12 ml:
(i) 2 ml of McCoy's 5A modified medium supplemented with 10% fetal bovine serum and 2 mM L-glutamine;
(ii) 2.0-5.0 ml mesenchymal stimulatory supplement; and
(iii) 2.0-5.0 ml methocult or mesencult medium.

21. The method of claim 16, wherein said progenitor stem cells are directed to obtain a lineage of differentiated cells.

22. The method of claim 21, directing and converting said progenitor stem cells into a lineage of immunoglobulin producing cells that secrete autologous antibodies to an antigen.

23. The method of claim 21, wherein said progenitor cells are cultured on a feeder layer of tumor cells to produce a lineage of cells producing antibodies against said tumor cells.

24. The method of claim 23, wherein said antibodies are specific against said tumor cells.


SEQUENCE LISTING (4 sequences)

SEQ ID NO: 1 - DNA, 21 bp, artificial sequence
  keratin19 3′ epithelial cells
catgagccgctggtactcctg

SEQ ID NO: 2 - DNA, 21 bp, artificial sequence
  keratin19 5′ epithelial cells
atcctgagtgacatgcgaagc

SEQ ID NO: 3 - DNA, 20 bp, artificial sequence
  beta-actin 5′ epithelial cells
tcaccaactgggacgacatg

SEQ ID NO: 4 - DNA, 20 bp, artificial sequence
  beta-actin 3′ epithelial cells
atgtcacgcacgatttcccg